(19) United States
(12) Patent Application Publication
(10) Pub. No.: US 2021/0259794 A1
(43) Pub. Date: Aug. 26, 2021

(54) Medical Apparatus Having Dual Manipulation Means and Methods of Use Thereof

(71) Applicants: Canon USA, Inc., Melvile, NY (US); The General Hospital Corporation, Boston, MA (US); The Brigham and Women's Hospital Inc., Boston, MA (US)

(72) Inventors: Takahisa Kato, Brookline, MA (US); Eric W Halvorson, Boston, MA (US); Matthew Michael Kincaid, Medford, MA (US); Brian Ninni, Brighton, MA (US); Charles George Hwang, Boston, MA (US); Yolonda Lorig Colson, Dover, MA (US); Hisashi Tsukada, Boston, MA (US)

(21) Appl. No.: 17/179,153

(22) Filed: Feb. 18, 2021

Related U.S. Application Data
(60) Provisional application No. 62/980,072, filed on Feb. 21, 2020.

(51) Int. Cl. A61B 34/00; A61B 17/00

(52) U.S. Cl. CPC A61B 34/74; A61B 2090/064; A61B 17/00234

(57) ABSTRACT

An articulated medical device having a hollow core, capable of large degrees of maneuverability through small cavities to reach a target with minimal invasiveness, wherein the medical device is capable of manual and robotic manipulation.

### CROSS REFERENCE TO RELATED PATENT APPLICATIONS

This application claims priority from U.S. Provisional Patent Application No. 62/980,072 filed on Feb. 21, 2020, in the United States Patent and Trademark Office, the disclosure of which is incorporated herein in its entirety by reference.

### FIELD OF THE DISCLOSURE

The present disclosure relates generally to apparatus and methods for medical application. More particularly, the subject disclosure is directed to an articulated medical device capable of manual and automatic manipulation in a patient, wherein the device has a hollow cavity for allowing a medical tool to be guided through the hollow cavity for medical procedures, including endoscopes, cameras, and catheters.

### BACKGROUND OF THE DISCLOSURE

Bendable medical instruments such as endoscopic surgical instruments and catheters are well known and continue to gain acceptance in the medical field. The bendable medical instrument generally includes a flexible body commonly referred to as a sleeves or sheaths. One or more tool channels extend along (typically inside) the flexible body to allow access to a target located at a distal end of the body.

The instrument is intended to provide flexible access within a patient, with at least one curve or more leading to the intended target, while retaining torsional and longitudinal rigidity so that a physician can control the tool located at the distal end of the medical instrument by maneuvering the proximal end of the instrument.

Recently, to enhance maneuverability of the distal end of the instrument, robotized instruments that control distal portions have emerged. In those robotized instruments, to create curves locally at the distal portion by robotics, different techniques have been disclosed.

By way of example, United States patent publication number 2016/0067450, provides multiple conduits to retain the shape of the proximal part, while the driving tendons are bending the distal part in the medical instruments. The multiple conduits would be controlled selectively in a binary way by constraining or unconstraining the proximal ends of the conduits. By selecting the constrained conduits, the bendable medical device can change the length of bending distal segment by changing the stiffness of the bendable medical device based on the area where the conduits deploy.

However, there remains a need in the industry to further refine and advance the methods for using the bendable medical devices, in that the time and flexibility associated with advancing the device into the patient can be lengthy, tedious, and may require constant attention by the physician to manually insert the device.

### SUMMARY

Thus, to address such exemplary needs in the industry, the presently disclosure provides a method for manipulating a medical apparatus comprising the steps of: providing the medical apparatus comprising: a bendable body having at least one bendable section; at least one control wire slideably situated in the bendable body and attached to a distal end of the bendable body; and an actuator connected to the at least one control wire and configured to actuate the control wire to manipulate the at least one bendable section, manipulating the medical apparatus manually using the actuator; providing an insertion unit configured to couple with the medical apparatus; connecting the medical apparatus to the insertion unit for robotic manipulation of the medical apparatus; and manipulating the medical apparatus robotically, wherein the manual manipulation of the medical device is configured to advance the medical device faster than the robotic manipulation of the medical device.

In various other embodiment, the method may further comprise having a controller in communication with the medical device, where in the controller is configured to robotically manipulate the at least one control wire.

In other embodiments, the actuator may be configured to manipulate the at least one bendable section when the medical apparatus is disconnected from the insertion unit.

It is also contemplated that the actuation unit may be configured to be held by an end user when the bendable body assembly is detached from the insertion unit.

In yet additional embodiments, the insertion unit further comprises a force sensors to measure a force applied to the bendable body.

In another embodiment, the medical apparatus further comprises a second bendable section, wherein a controller is configured to control one bending section directly associated with an end user input, and to control the second bending section associated with an algorithm in the controller.

It is further contemplated that the controller controls the most distal bendable section directly associated with an end user input, and controls the other bendable section with an algorithm in the control mode.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 illustrates an exemplary bendable medical device incorporating various ancillary components, according to one or more embodiment of the subject apparatus, method or system.

FIG. 2A provides a partial depiction of an exemplary bendable medical device, with the device mounted to an insertion unit, according to one or more embodiment of the subject apparatus, method or system.

FIG. 2B provides a partial depiction of an exemplary bendable medical device, with the device separated from the insertion unit, according to one or more embodiment of the subject apparatus, method or system.

FIG. 3 depicts a perspective view of an exemplary bendable medical device, according to one or more embodiment of the subject apparatus, method or system.

FIG. 4 provides an internal view of an exemplary bendable medical device, according to one or more embodiment of the subject apparatus, method or system.

FIGS. 5A-5C provide side perspective views of an exemplary bendable medical device at various bendable orientations, according to one or more embodiment of the subject apparatus, method or system.

FIG. 6 depicts a cut-away view of an exemplary bendable medical device navigating through a tortuous pathway, according to one or more embodiment of the subject apparatus, method or system.

FIG. 7 provides a flow chart detailing a method for employing an exemplary bendable medical device, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

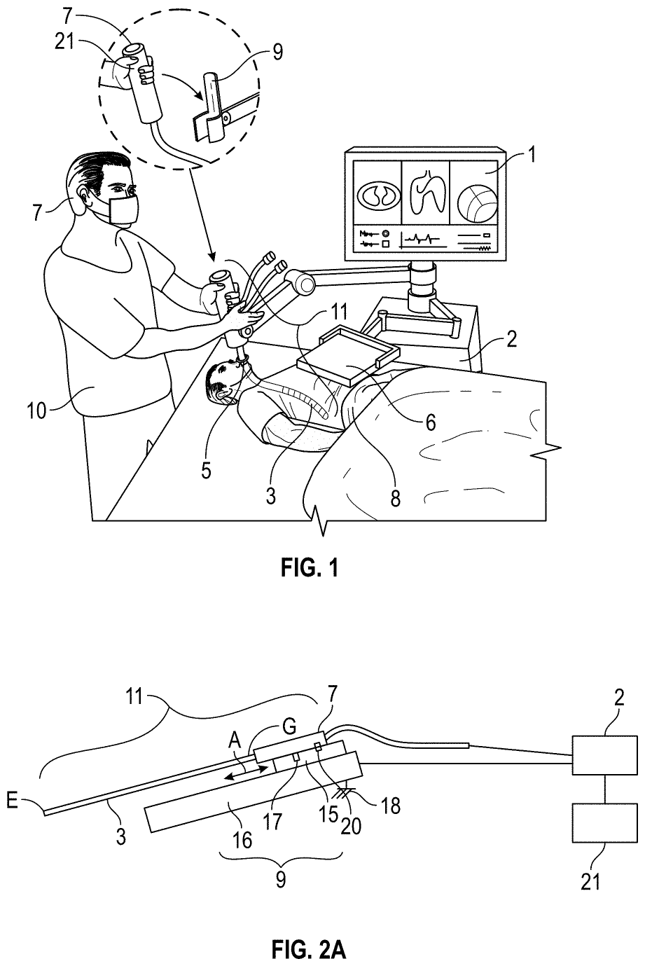

FIG. 1 illustrates an exemplary bendable medical device 11 incorporating various ancillary components, according to one or more embodiment of the subject apparatus, method or system. The bendable medical device 11 is in communication with navigation software on a computer 1, as well as a controller 2 which are communicatively-coupled via a bus to transmit/receive data between each other. Moreover, the navigation software and computer 1 are connected to and communicates with a CT scanner, a fluoroscope and an image server (not in Figure), which are external of the bendable medical device 11. The image server includes but is not limited to a DISCOM™ server connected to a medical imaging device 11 including but not limited to a CT and/or MRI scanner and a fluoroscope. The navigation software 1 processes data provided by the controller 2 and data provided by images stored on the image server, and/or images from the CT scanner and the fluoroscope in order to display images onto the image display.

The images from the CT scanner are pre-operatively provided to the navigation software 1. With the navigation software 1, an end user may create an anatomical computer model from the images. In this particular embodiment, the anatomy examined are the lungs. From the chest images of the CT scanner, the clinical user can segment the lung airways for various clinical treatments, such as a biopsy. After generating the lung airway map, the user can also create a plan for accessing the lesion for the biopsy. The plan includes the pathway for insertion of the bendable body 3 of the bendable medical device 11, through the airways to arrive at the lesion. The controller 2 includes a control circuitry, which is communicatively-coupled with the actuation unit 7, insertion unit 9, field generator 6 and man-machine interface 21 (not in Figure) for manipulating those units.

The bendable body 3 is attached with the actuation unit 7 and forms the bendable medical device 11. The actuation unit 7 is configured to bend various bending sections of the bendable body 3 with the controller 2. The bendable medical device 11 is detachably attached to the insertion unit 9, thus allowing for the bendable medical device 11 to be stationary. The controller 2 can configured to synchronize behavior of actuation unit 7 with behavior of insertion unit 9, and can change those behaviors based on whether the bendable medical device 11 is attached to the insertion unit 9 (hereafter “on-state”) or detached from the insertion unit 9 (hereafter “off-state”).

The bendable body 3 is configured to be inserted into the lungs of patient 8. In an exemplary embodiment, the physician 10 will facility the input value(s) to the controller 2, via the man-machine interface 21 (ex. Joystick), and the controller 2 will move the actuation unit 7 and/or insertion unit 9 to reflect the physician's 10 intended movement of the bendable body 3. The bendable body 3 advances to the target lesion, where a medical tool (ex. Biopsy tool) may be guided by using tool channel in the bendable body 3.

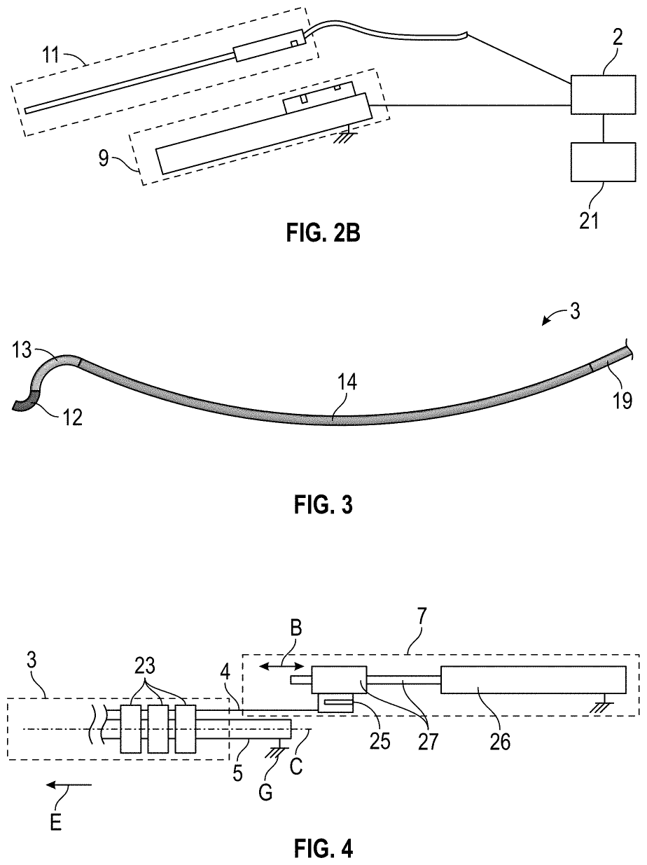

FIGS. 2A and 2B is illustrate the two configurations of the bendable medical device 11, namely, on-state and off-state. In the on-state configuration shown in FIG. 2A, the bendable medical device 11 is mounted and locked on the insertion unit 9 with a detachable lock 20. The lock 20 may be unlocked manually with a mechanical unlock structure or with an electrical unlock structure incorporating the controller 2. An attachment sensor 17 may be configured on the insertion unit 9 and/or bendable body 3, to detect whether the bendable body assembly 11 is in the on or off state. An insertion slider 15, moving along line A, may be used to guide the bendable body 3 based on the command from the controller 2. The bendable body 3 has a distal end and a proximal end, represented by E and G, respectively in FIG. 2A.

In the on-state configuration, the physician 10 will control the insertion slider 15 and/or bendable body 3 with man-machine interface 21.

In the off-state configuration, provided in FIG. 2B, the bendable medical device 11 is detached from insertion slider 15 and insertion unit 9 all together. In this configuration, the physician 10 retains the actuation unit 7 with his/her hand(s), and can manipulate the bendable body 3 with either the man-machine interface 21 via controller 2, or can manually manipulate the bendable body 3.

This ability to switch from the on-state configuration to the off-state configuration, and vise-versa, allows for flexible adaptation of the bendable medical device 11, depending on the needed circumstances. For instance, during initial advancement of the bendable medical device 11 through larger/wider portions of a patient's anatomy, the off-state configuration can been implemented to encourage rapid advancement of the bendable medical device 11, thus saving time. Once the bendable medical device 11 reaches more tortuous sections of the patient's anatomy, the bendable medical device 11 may be switched to the on-state configuration, allowing for more finite advancement of the bendable medical device 11, which are done at a slower deliberate pace so as to ensure minimal abrasion and discomfort to the patient.

As detailed above, the subject method of use of the medical device 11 allows one to use the bendable body assembly both with and without the insertion unit 9. Robotic insertion control with the insertion unit 9 can provide a user with precise and consistent controls of insertion and bending motion of the bendable body by synchronizing the insertion and bending motions with the control of the bendable body assembly with the insertion unit. Besides, the control of the bendable body assembly without the insertion unit can provide a user with agile and flexible insertion operation while the user still leverages the robotic bending control, which significantly enhances safety and flexibility in advancing and retracting the medical device.

The user is allowed to have tactile feedback regarding the insertion of the bendable body so that the user can adjust insertion speed and force intuitively and quickly, as would be expected in the conventional manual insertion practice. However, the duration time for the procedure can be shortened while maintaining the same level of safety as the conventional manual insertion methods. As the subject innovation can reduce the insertion length with robotic insertion control, by allowing for manual insertion, the insertion unit can be miniaturized with a shorter stroke, which further leads to a lower cost for manufacture.

Further advantages include using force information in the operation mode when the sheath is detached from the insertion unit, wherein the controller can assist in bending the sheath with control of forces that the sheath would apply to the anatomy, while the user manually perform the insertion. Specifically, this is advantageous when the user controls a part of the degrees of freedom of motion in the sheath among the many degrees of freedom of motion to control.

By controlling the most distal bending section directly with the controller, the user can maintain the same maneuvers for the procedure as the conventional endoscope's. Also, since the controller can assist the rest of the degree of the freedom, the user can leverage additional degree of the freedom to improve the dexterity with the simple and minimal control parameters.

FIG. 3 is a schematic drawing to explain the bendable segments of the bendable medical device 3. The bendable medical device 3 comprises a proximal part 19 and three bendable segments, which are the first, second, and third bendable segments 12, 13, 14, respectively. The bendable segments 12, 13, 14, can independently bend and can form a shape with three independent curvatures.

FIG. 4 explains the bending principles of the bendable medical device 11, incorporating one or more driving wire(s) 4. The actuation unit 7 comprises motor 26, linear motion structure 27, and a force sensor 25. The motor 26 is configured to move the liner motion structure 27 to create linear motion along line B, both fore and aft. The force sensor 25 is also attached to the actuation unit 7, and will be moved together with the linear motion structure 27, thus allowing for accurate force readings when the linear motion structure 27 encounters resistance. The driving wire 4 is terminated on the linear motion structure 27, and is pushed and pulled along line B. Another end of driving wire 4 is terminated on the distal end of one of bending sections, situated slidingly through the wire guides 23. As the linear motion structure 27 is pushed and pulled by the motor 26, the bending section where this end of the driving wire 4 is terminated, is subjected to bending moment and can be bent according to the linear motion structure 27. The force sensor 25 can sense the force applied to driving wire 4, as well as the resistance for further calculations and amendments in force, if need be.

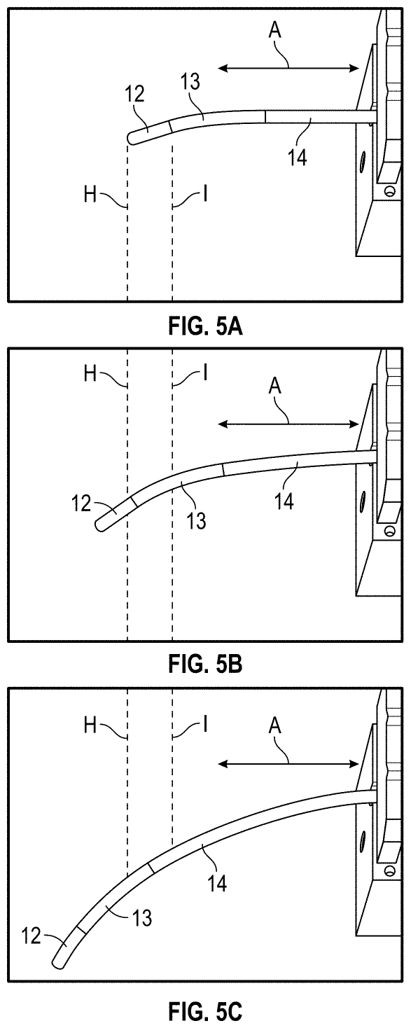

FIGS. 5A-5C detail exemplary embodiments of unique motion of the bendable medical device, with three bending sections 12, 13 and 14, and the insertion unit 9, incorporating follow-the-leader (FTL) motion. In the depictions provided, the physician may first issue command to bend distal bending section 12 (FIG. 5A). After that, the physician may advance bendable body 3 with the insertion unit 9 along line A. When the distal part of middle section 13 reaches the position of distal bending section 12 at previous moment shown in FIG. 5A (position H-I), the controller 2 now automatically bends the middle section 12 to the same bending angles as the distal bending section 12 at that position, much like follow the leader, which is where the name originates from. After that, the physician can advance the bendable body 3 further with the insertion unit 9 along line A (FIG. 5c). Upon advancement, when the distal part of the proximal bending section 14 reaches position H-I, the controller once again also bend the proximal bending section 14 to mimic the bending angles of bending sections 12 and 13, at that position (H-I).

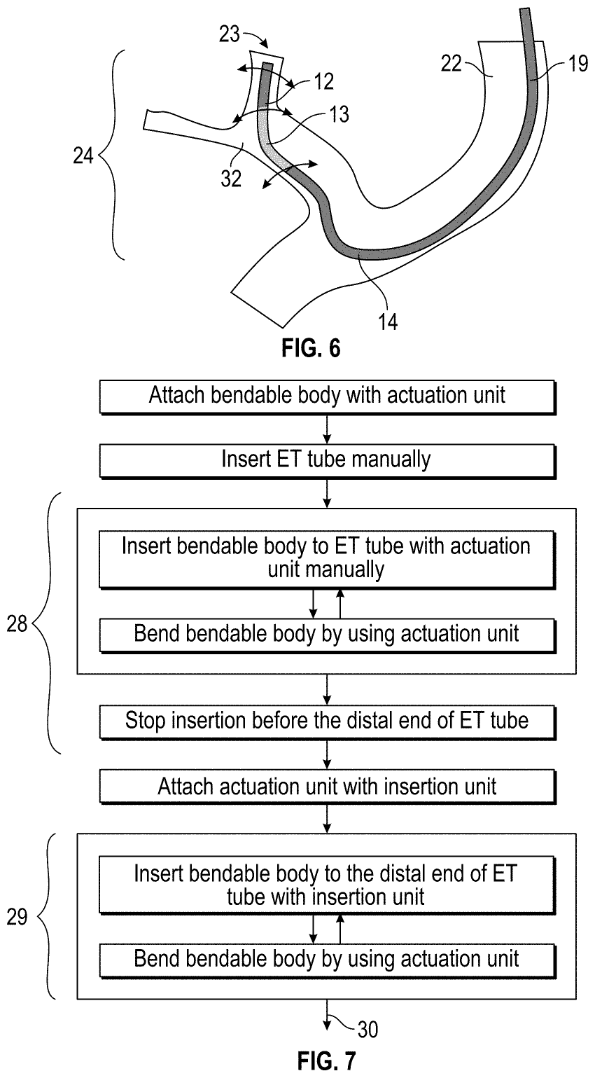

FIG. 6 provides a cut-away view of an exemplary bendable medical device 11 inserted into a cavity, specifically, the peri-bronchial area of a patient's lungs, which is a lateral area surrounding the airways. This area is a known challenge to target as identified in literature, and the prior art, due to the limited distal dexterity of the conventional catheter. To reach the lesion through airways 22 in the navigation stage, the first and the second bendable segments 12, 13, respectively, navigate the bendable medical device 11 through the bifurcation point 32. The first bendable segment 12 can adjust the shape/orientation to the daughter branch while the second bendable segment 13 can adjust the shape/orientation to the parent branch in the bifurcation point 32, as the bendable medical device 11 advances through the bifurcation point 32. Once the first and the second bendable segments 12 and 13 pass the bifurcation point 32, those segments may act as guides for the rest of the bendable medical device 11, so that the insertion force from the proximal end of the bendable medical device 1 can be effectively transformed into the insertion force for a distal part of the bendable medical device 11 without serious prolapsing of the distal section. Once the distal end 34 of the bendable medical device 11 reaches the vicinity of the lesion 23, the bendable medical device 3 would direct the distal end 24 to a lesion 36, which locates the lateral area around the airway, by bending the first and the second bendable segments 12 and 13, respectively. Since the airway doesn't directly connect with the lesion 36, this is one of the more difficult configurations for a conventional catheter.

With the first, the second and the third bendable segments 12, 13 and 14, respectively, the bendable medical device 11 can orient the distal end 34 without moving the proximal part 19 that goes through all bifurcations to this lesion 36. By using the three-dimensional bending capability of the first and the second bendable segments 12 and 13, the bendable medical device 11 can perform unique maneuvers to enhance capability of the peri-bronchial targeting. Therefore, the bendable medical device 11 can provide improved access to the intended lesion 36 through tortuous pathways. Also, the bendable medical device 11 can have different flexibility along the axial direction without increasing the size or number of the jointing points.

FIG. 7 is the workflow diagram showing an exemplary transition from manual insertion of the bendable medical device 11 incorporating robotic steering 28 in the off-state configuration, to robotic insertion of the bendable medical device 11 incorporating robotic steering 29 in the on-state configuration.

At this time, the bendable medical device 11 has been advanced into the patient's body, and also has endoscope unit in the tool channel to use endoscopic view. Then, the physician 10 places an endobronchial tube (ET tube) into the patient. Following placement of ET tube, the physician 10 inserts the bendable medical device 11 into the ET tube manually by manipulating the actuation unit 7 by hand. At the same time, the physician 10 can steer the distal bending section 12 with actuation unit 7 by utilizing the endoscope view. Once the physician 10 inserts the bendable medical device 11 close to the distal edge of ET tube, the physician 10 halts manual insertion.

At this junction, the physician 10 attaches the bendable medical device 11 to the insertion unit 9. Upon attachment and proper locking of the lock 20, the attachment sensor 17 is engaged with the controller 2, and the bendable medical device 11 transitions from the off-state configuration to the on-state configuration, which activates FTL motion mode. The physician 10 controls the insertion unit 9 and distal bending section 12 with actuation unit 7, and inserts bendable body 3 with FTL motion.

Once the physician 10 reaches the distal edge of the ET tube by confirming endoscopic view, the physician 10 can now teach the insertion slider positon as the position of distal edge of ET tube to tell the controller 2 the maximum insertion depth that the physician 10 requires for this procedure. After that, the physician 10 may continue to use FTL motion to insert the bendable device 11 for airway navigation 30.

While the present disclosure has been described with reference to exemplary embodiments, it is to be understood that the disclosure is not limited to the sample exemplary embodiments provided herein. The scope of the following claims is to be accorded the broadest interpretation so as to encompass all such modifications and equivalent structures and functions.

1. A method for manipulating a medical apparatus comprising the steps of:
providing the medical apparatus comprising:
a bendable body having at least one bendable section;
at least one control wire slideably situated in the bendable body and attached to a distal end of the bendable body; and
an actuator connected to the at least one control wire and configured to actuate the control wire to manipulate the at least one bendable section, manipulating the medical apparatus manually;
providing an insertion unit configured to couple with the medical apparatus; and
manipulating the medical apparatus robotically,
wherein the manual manipulation of the medical device is configured to advance the medical device faster than the robotic manipulation of the medical device.

2. The method of claim 1, further comprising a controller in communication with the medical device, configured to robotically manipulate the at least one control wire.

3. The method of claim 1, wherein the actuator is configured for manual manipulation of the medical apparatus, when disconnected from the insertion unit.

4. The method of claim 1, wherein the actuation unit is configured to be held by an end user when the bendable body assembly is detached from the insertion unit.

5. The method of claim 1, wherein the insertion unit further comprises at least one force sensor to measure a force applied to the bendable body.

6. The method of claim 1, further comprising a second bendable section, wherein a controller is configured to control one bending section directly associated with an end user input, and to control the second bending section associated with an algorithm in the controller.

7. The method of claim 6, wherein the controller controls the most distal bendable section directly associated with an end user input, and controls the other bendable section with an algorithm in the control mode.

8. The method of claim 1, further comprising connecting the medical apparatus to the insertion unit for robotic manipulation of the medical apparatus.
